(19) United States
(12) Patent Application Publication
(10) Pub. No.: US 2023/0414920 A1
(43) Pub. Date: Dec. 28, 2023

(54) INTRAVASCULAR BLOOD PUMPS AND METHODS OF USE

(71) Applicant: SHIFAMED HOLDINGS, LLC, Campbell, CA (US)

(72) Inventors: Amr SALAHIEH, Saratoga, CA (US); Janine ROBINSON, Half Moon Bay, CA (US)

(21) Appl. No.: 18/247,715

(22) PCT Filed: Oct. 4, 2021
(86) PCT No.: PCT/US21/53433

§ 371 Date: Apr. 3, 2023

Related U.S. Application Data
(60) Provisional application No. 63/086,791, filed on Oct. 2, 2020.

(51) Int. Cl. A61M 60/13; A61M 60/808

(52) U.S. Cl. CPC A61M 60/13; A61M 60/808

(57) ABSTRACT

An intravascular blood pump having an expandable blood conduit with an unexpanded delivery diameter and an expanded deployed diameter; one or more expandable impellers disposed at least partially in the blood conduit; and an inflatable member disposed distal to the blood conduit, the inflatable member having an uninflated delivery configuration and an inflated configuration, the inflatable member having a diameter in the inflated configuration that is greater than the expanded deployed diameter of the blood conduit.

### CROSS REFERENCE TO RELATED APPLICATIONS

This application claims the benefit of U.S. Provisional Patent Application No. 63/086,791, filed Oct. 2, 2020, the disclosure of which is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Intravascular blood pumps may be positioned at an anatomical location in which a pump inflow is proximate or near to tissue. It may be desirable to prevent the tissue from being pulled towards or into the pump inflow during operation of the pump. Additionally or alternatively, it may be desirable to space or distance the pump inflow from the tissue to help prevent the tissue from being pulled towards the inflow and interfering with pump performance.

### SUMMARY OF THE DISCLOSURE

The present invention relates to an inflatable member that may be used to space the inlets of an expandable intravascular blood pump from the patient's tissue. The inflatable member may be, e.g., disposed at the distal end of the blood pump.

One aspect of the invention provides an intravascular blood pump having an expandable blood conduit with an unexpanded delivery diameter and an expanded deployed diameter; one or more expandable impellers disposed at least partially in the blood conduit; and an inflatable member disposed distal to the blood conduit, the inflatable member having an uninflated delivery configuration and an inflated configuration, the inflatable member having a diameter in the inflated configuration that is greater than the expanded deployed diameter of the blood conduit.

In some embodiments, the one or more impellers include an impeller disposed at a distal end of the blood conduit.

In either or both of these embodiments, the intravascular blood pump also has a guidewire lumen extending through the inflatable member. In some such embodiments, the intravascular blood pump also has an elongate member extending distally from a hub at a distal end of the blood conduit, the guidewire lumen extending through the hub and the elongate member. The elongate member may optionally be more flexible than the hub.

In some embodiments, the inflatable member includes an inelastic material. The inflatable member may have a preformed shape, such as spherical, ovoid, or toroidal.

In some embodiments, the inflatable member is adapted and configured to be partially inflated during intravascular delivery to seal a distal end of a delivery sheath.

Another aspect of the invention provides a catheter-based intravascular blood pump, having a pump portion including a blood conduit, one or more impellers disposed at least partially within the blood conduit, and a distal pump region that is distal to the blood conduit; and an inflatable member transitionable between an uninflated state suitable for delivery and an inflated state, the inflatable member secured to the distal pump region, the inflatable member having a configuration in the inflated state to space the distal pump region from tissue adjacent the distal pump region.

In some embodiments of this aspect of the invention, one or more of a distal spacing from the blood conduit and the configuration in the inflated state prevent tissue from being pulled into the pump portion inflow during pump operation.

In some embodiments, the inflatable member has an internal volume adapted to in be fluid communication with an external fluid source. In some such embodiments, the inflatable member has an internal volume adapted to in be fluid communication with a purge fluid source, the catheter blood pump having at least one fluid pathway therein (optionally a fluid lumen) in fluid communication with the internal volume. In some such embodiments, the fluid pathway includes a rotatable drive mechanism (e.g., drive cable, drive shaft, or a combination thereof) in operable communication with the one or more impellers. The fluid pathway may also be a fluid pathway in fluid communication with a distal bearing disposed proximate the blood inflow.

In any of these embodiments, the inflatable member (optionally a proximal region thereof) is coupled to a central hub to which a plurality of distal pump struts are coupled.

In these embodiments, a rotatable drive mechanism may be disposed within the central hub, the pump portion optionally comprising a distal bearing surrounding the rotatable drive mechanism.

In some embodiments, the inflatable member (optionally a distal region thereof) is coupled to an elongate element extending distally from the central hub, optionally wherein the elongate element is more flexible than the central hub.

In some embodiments, the inflatable member is coupled to an axially extending element that forms part of a guidewire lumen.

The inflatable member may be made of an elastic material, such as a polymeric material. Alternatively or additionally, the inflatable member may include an inelastic material, and it may be optionally preformed in the inflated state so that inflation of the internal volume inflates the inflatable member toward the pre-formed shape (e.g., spherical, ovoid, toroidal). In some embodiments, the pre-formed shaped has a proximal region with a surface configured to facilitate flow towards the pump inflow, such as a curved surface having a radially outer dimension decreasing in a proximal direction. The proximal region may have a concave surface.

In any such embodiments, the inflatable member may have a plurality of outflow apertures therein. The inflatable member may be a weeping balloon.

In some embodiments, the inflatable member has a proximal end that is attached to a component that is not unitary with a second component to which an inflatable member distal end is attached.

In some embodiments, the blood conduit, and optionally the one or more impellers, are adapted to be transitioned between an expanded configuration and a collapsed configuration, optionally with an outer sheath. Alternatively, in other embodiments the blood conduit and the one or more impellers are non-expandable and non-collapsible such are not adapted to be transitioned between an expanded configuration and a collapsed configuration.

In any of the these embodiments, the inflatable member may be sized and configured and positioned axially to prevent blood from entering the distal end of a delivery sheath during tracking and delivery of the intravascular blood pump through the patient's vasculature. In some such embodiments, the inflatable member may have a partially inflated state configured to prevent blood from entering the distal end of a delivery sheath for tracking and delivery.

Another aspect of the invention provides a catheter-based intravascular blood pump having a pump portion including a blood conduit and one or more impellers disposed at least partially within the blood conduit; and an inflatable member transitionable between an uninflated configuration suitable for delivery and an inflated configuration, the inflatable member spaced distally from a pump portion inflow.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows the distal end of an intravascular blood pump according to one embodiment of the invention.

FIG. 2 shows use of the intravascular blood pump in the embodiment of FIG. 1 to space pump inlets away from a patient's tissue.

FIG. 3 shows the distal end of an intravascular blood pump according to another embodiment of the invention.

FIG. 4 shows advancement of an intravascular blood pump and a delivery sheath with an inflatable member disposed at the distal end of the delivery sheath.

FIG. 5 is a side view of an exemplary pump portion of a catheter-based intravascular blood pump that includes a conduit, a plurality of impellers, an expandable member

FIG. 6 is a side view of an exemplary pump portion that includes a conduit, a plurality of impellers, and a plurality of expandable members.

FIGS. 7A-D illustrate an exemplary pump portion that includes a conduit, a plurality of impellers, and a plurality of expandable members.

FIG. 8 illustrates an exemplary pump portion.

FIGS. 9A-D illustrate an exemplary blood pump that includes a guidewire pathway and at least one fluid purge pathway.

FIGS. 10A and B illustrate an exemplary blood pump that includes a guidewire pathway and at least two fluid purge pathways that are not in fluid communication.

### DETAILED DESCRIPTION

The disclosure herein is related to intravascular blood pumps, their methods of use and manufacture. Blood pumps herein may include one or more inflatable members distal to a blood conduit and proximate a pump inflow. The inflatable members herein generally are secured to a distal pump region and configured to maintain a spacing between the distal pump region and tissue adjacent the distal pump region, such as a left ventricle wall. The inflatable member may be spaced distally from the blood conduit and may have a configuration in the inflated state to prevent tissue from being pulled into the pump inflow during operation of the pump. The pump portions herein may be placed in different anatomical locations and the inflatable members herein may still provide the same benefits described herein.

The inflatable members described herein may be incorporated into a variety of blood pumps having one, two, or more expandable impellers disposed in a variety of expandable housings or shrouds, such as those described in any of WO2018/226991; WO2019/094963; WO2019/152875; WO2020/028537; and WO2020/073047. For example, FIG. 5 is a side view illustrating a distal portion of an exemplary intravascular blood pump, including pump portion 1600, wherein pump portion 1600 includes an expandable proximal impeller 1606 and an expandable distal impeller 1616, both of which are in operable communication with drive cable 1612. Pump portion 1600 is in an expanded configuration in FIG. 5, but is adapted to be collapsed to a delivery configuration so that it can be delivered with a lower profile. The impellers can in rotational communication with drive cable 1612, directly or indirectly. Drive cable 1612 is in operable communication with an external motor, not shown, and extends through elongate shaft 1610. The phrases “pump portion” and “working portion” (or derivatives thereof) may be used herein interchangeably unless indicated to the contrary. For example, without limitation, “pump portion” 1600 can also be referred to herein as a “working portion.”

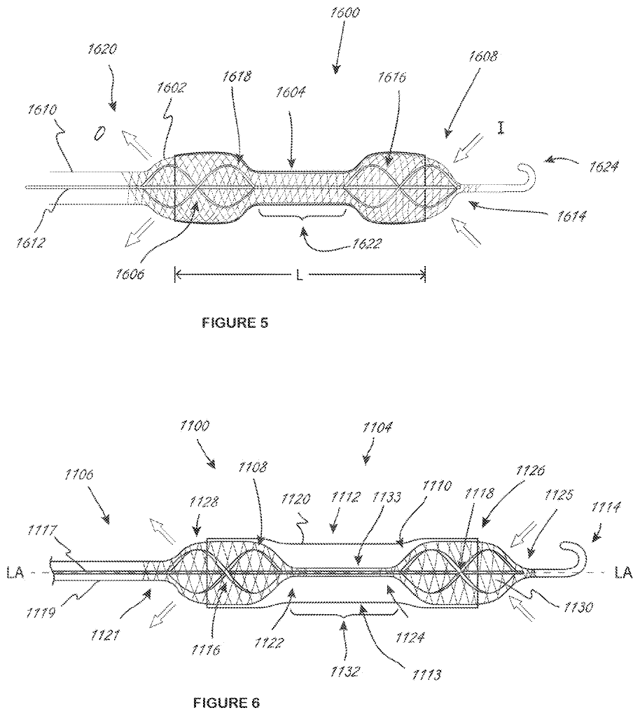

FIG. 6 is a side view illustrating a deployed configuration (shown extracorporeally) of a distal portion of an exemplary embodiment of an intravascular blood pump. Exemplary system 1100 includes pump portion 1104 (which as set forth herein may also be referred to herein as a pump portion) and an elongate portion 1106 extending from pump portion 1104. Elongate portion 1106 can extend to a more proximal region of the system, not shown for clarity, and that can include, for example, a motor. Pump portion 1104 includes first expandable member 1108 and second expandable member 1110, axially spaced apart along a longitudinal axis LA of pump portion 1104. Spaced axially in this context refers to the entire first expandable member being axially spaced from the entire second expandable member along a longitudinal axis LA of pump portion 1104. A first end 1122 of first expandable member 1108 is axially spaced from a first end 1124 of second expandable member 1110. Some “expandable members” herein may also be referred to herein as baskets, housings, or shrouds.

First and second expandable members 1108 and 1110 generally each include a plurality of elongate segments disposed relative to one another to define a plurality of apertures 1130, only one of which is labeled in the second expandable member 1110. The expandable members can have a wide variety of configurations and can be constructed in a wide variety of ways, such as any of the configurations or constructions in, for example without limitation, U.S. Pat. No. 7,841,976, or the tube in U.S. Pat. No. 6,533,716, which is described as a self-expanding metal endoprosthetic material. For example, without limitation, one or both of the expandable members can have a braided construction or can be at least partially formed by laser cutting a tubular element.

Pump portion 1104 also includes blood flow conduit 1112, which in this embodiment is supported by first expandable member 1108 and to second expandable member 1110. Conduit 1112 also extends axially in between first expandable member 1108 and second expandable member 1110 in the deployed configuration. A central region 1113 of conduit 1112 spans an axial distance 1132 where the pump portion is void of first and second expandable members 1108 and 1110. Central region 1113 can be considered to be axially in between the expandable members. Distal end 1126 of conduit 1112 does not extend as far distally as a distal end 1125 of second expandable member 1110, and proximal end of conduit 1128 does not extend as far proximally as proximal end 1121 of first expandable member 1108.

When the disclosure herein refers to a conduit being coupled to an expandable member, the term coupled in this context does not require that the conduit be directly attached to the expandable member so that conduit physically contacts the expandable member. Even if not directly attached, however, the term coupled in this context refers to the conduit and the expandable member being joined together such that as the expandable member expands or collapses, the conduit also begins to transition to a different configuration and/or size. Coupled in this context therefore refers to conduits that will move when the expandable member to which it is coupled transitions between expanded and collapsed configurations. The conduits herein are considered to create a pathway for fluid to be moved, and may be defined by a one or more components of the pump portion.

Any of the conduits herein can be deformable to some extent. For example, conduit 1112 includes elongate member 1120 that can be made of one or more materials that allow the central region 1113 of conduit to deform to some extent radially inward (towards LA) in response to, for example and when in use, forces from valve tissue (e.g., leaflets) or a replacement valve as pump portion 1104 is deployed towards the configuration shown in FIG. 6. The conduit may be stretched tightly between the expandable members in some embodiments. The conduit may alternatively be designed with a looseness that causes a greater degree of compliance. This can be desirable when the pump portion is disposed across fragile structures such as an aortic valve, which may allow the valve to compress the conduit in a way that minimizes point stresses in the valve. In some embodiments, the conduit may include a membrane attached to the proximal and distal expandable members. Exemplary materials that can be used for any conduits herein include, without limitations, polyurethane rubber, silicone rubber, acrylic rubber, expanded polytetrafluoroethylene, polyethylene, polyethylene terephthalate, including any combination thereof.

Any of the conduits herein can have a thickness of, for example, 0.5-20 thousandths of an inch (thou), such as 1-15 thou, or 1.5 to 15 thou, 1.5 to 10 thou, or 2 to 10 thou.

Any of the conduits herein, or at least a portion of the conduit, can be impermeable to blood. In FIG. 6, pump portion 1104 includes a lumen that extends from distal end 1126 of conduit 1112 and extends to proximal end 1128 of conduit 1112. The lumen is defined by conduit 1112 in central region 1113, but can be thought of being defined by both the conduit and portions of the expandable members in regions axially adjacent to central region 1113. In this embodiment, however, it is the conduit material that causes the lumen to exist and prevents blood from passing through the conduit.

Any of the conduits herein that are secured to one or more expandable members can be, unless indicated to the contrary, secured so that the conduit is disposed radially outside of one or more expandable members, radially inside of one or more expandable members, or both, and the expandable member can be impregnated with the conduit material.

The proximal and distal expandable members help maintain the conduit in an open configuration by providing radial support for the conduit, while each also creates a working environment for an impeller, described below. Each of the expandable members, when in the deployed configuration, is maintained in a spaced relationship relative to a respective impeller, which allows the impeller to rotate within the expandable member without contacting the expandable member. Pump portion 1104 includes first expandable impeller 1116 and a second expandable impeller 1118, with first impeller 1116 disposed radially within first expandable member 1108 and second impeller 1118 disposed radially within second expandable member 1110. In this embodiment, the two impellers even though they are distinct and separate impellers, are in operable communication with a common drive mechanism (e.g., drive cable 1117), such that when the drive mechanism is activated the two impellers rotate together. In this deployed configuration, impellers 1116 and 1118 are axially spaced apart along longitudinal axis LA, just as are the expandable members 1108 and 1110 are axially spaced apart.

Impellers 1116 and 1118 are also axially within the ends of expandable members 1108 and 1110, respectively (in addition to being radially within expandable members 1108 and 1110). The impellers herein can be considered to be axially within an expandable member even if the expandable member includes struts extending from a central region of the expandable member towards a longitudinal axis of the pump portion (e.g., tapering struts in a side view). In FIG. 6, second expandable member 1110 extends from first end 1124 (proximal end) to second end 1125 (distal end).

In FIG. 6, a distal portion of impeller 1118 extends distally beyond distal end 1126 of conduit 1112, and a proximal portion of impeller 1116 extends proximally beyond proximal end 1128 of conduit 1112. In this figure, portions of each impeller are axially within the conduit in this deployed configuration.

In the exemplary embodiment shown in FIG. 6, impellers 1116 and 1118 are in operable communication with a common drive mechanism 1117, and in this embodiment, the impellers are each coupled to drive mechanism 1117, which extends through shaft 1119 and pump portion 1104. Drive mechanism 1117 can be, for example, an elongate drive cable, which when rotated causes the impellers to rotate. In this example, as shown, drive mechanism 1117 extends to and is axially fixed relative to distal tip 1114, although it is adapted to rotate relative to distal tip 1114 when actuated. Thus, in this embodiment, the impellers and drive mechanism 1117 rotate together when the drive mechanism is rotated. Any number of known mechanisms can be used to rotate drive mechanism, such as with a motor (e.g., an external motor).

The expandable members and the conduit are not in rotational operable communication with the impellers and the drive mechanism. In this embodiment, proximal end 1121 of proximal expandable member 1108 is coupled to shaft 1119, which may be a shaft of elongate portion 1106 (e.g., an outer catheter shaft). Distal end 1122 of proximal expandable member 1108 is coupled to central tubular member 1133, through which drive mechanism 1117 extends. Central tubular member 1133 extends distally from proximal expandable member 1108 within conduit 1112 and is also coupled to proximal end 1124 of distal expandable member 1110. Drive mechanism 1117 thus rotates within and relative to central tubular member 1133. Central tubular member 1133 extends axially from proximal expandable member 1108 to distal expandable member 1110. Distal end 1125 of distal expandable member 1110 is coupled to distal tip 1114, as shown. Drive mechanism 1117 is adapted to rotate relative to tip 1114, but is axially fixed relative to tip 1114.

Pump portion 1104 is adapted and configured to be collapsed to a smaller profile than its deployed configuration (which is shown in FIG. 6). This allows it to be delivered using a lower profile delivery device (smaller French size) than would be required if none of pump portion 1104 was collapsible. Even if not specifically stated herein, any of the expandable members and impellers may be adapted and configured to be collapsible to some extent to a smaller delivery configuration.

The pump portions herein can be collapsed to a collapsed delivery configuration using conventional techniques, such as with an outer sheath that is movable relative to the pump portion (e.g., by axially moving one or both of the sheath and pump portion). For example without limitation, any of the systems, devices, or methods shown in the following references may be used to facilitate the collapse of a pump portion herein: U.S. Pat. No. 7,841,976 or U.S. Pat. No. 8,052,749, the disclosures of which are incorporated by reference herein for all purposes.

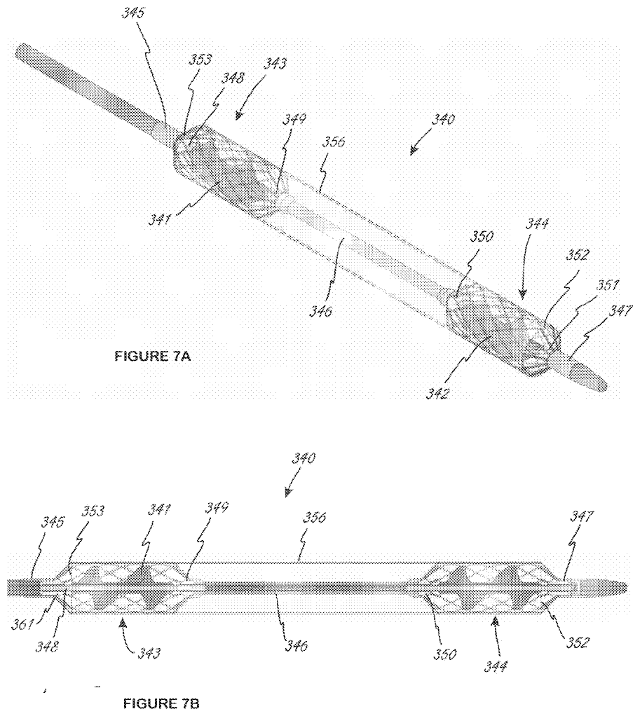

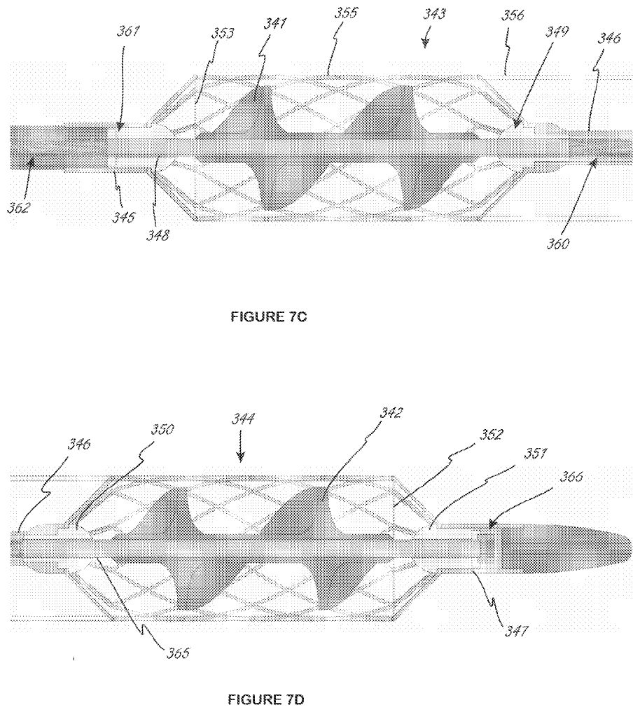

FIGS. 7A-D show an exemplary pump portion that is similar in some ways to the pump portion shown in FIG. 6. Pump portion 340 is similar to pump portion 1104 in that in includes two expandable members axially spaced from one another when the pump portion is expanded, and a conduit extending between the two expandable members. FIG. 7A is a perspective view, FIG. 7B is a side sectional view, and FIGS. 7C and 7D are close-up side sectional views of sections of the view in FIG. 7B.

Pump portion 340 includes expandable proximal impeller 341 and expandable distal impeller 342, which are coupled to and in operational communication with a drive cable, which defines therein a lumen. The lumen can be sized to accommodate a guidewire, which can be used for delivery of the pump portion to the desired location. The drive cable, in this embodiment, includes first section 362 (e.g., wound material), second section 348 (e.g., tubular member) to which proximal impeller 341 is coupled, third section 360 (e.g., wound material), and fourth section 365 (e.g., tubular material) to which distal impeller 342 is coupled. The drive cable sections all have the same inner diameter, so that lumen has a constant inner diameter. The drive cable sections can be secured to each other using known attachment techniques. A distal end of fourth section 365 extends to a distal region of the pump portion, allowing the pump portion to be, for example, advanced over a guidewire for positioning the pump portion. In this embodiment the second and fourth sections can be stiffer than first and third sections. For example, second and fourth can be tubular and first and third sections can be wound material to impart less stiffness.

Pump portion 340 includes a blood flow conduit, proximal expandable member 343 and distal expandable member 344, each of which extends radially outside of one of the impellers. The expandable members have distal and proximal ends that also extend axially beyond distal and proximal ends of the impellers, which can be seen in FIGS. 7B-7D. That pumps also includes conduit 356, which has a proximal end 353 and a distal end 352. The two expandable members each include a plurality of proximal struts and a plurality of distal struts. The proximal struts in proximal expandable member 343 extend to and are secured to shaft section 345, which is coupled to bearing 361, through which the drive cable extends and is configured and sized to rotate. The distal struts of proximal expandable member 343 extend to and are secured to a proximal region (to a proximal end in this case) of central tubular member 346, which is disposed axially in between the expandable members. The proximal end of central tubular member 346 is coupled to bearing 349, as shown in FIG. 7C, through which the drive cable extends and rotates. The proximal struts of distal expandable member 344 extend to and secured to a distal region (to a distal end in this case) of central tubular member 346. Bearing 350 is also coupled to the distal region of central tubular member 346, as is shown in FIG. 7D. The drive cable extends through and rotates relative to bearing 350. Distal struts of distal expandable member extend to and are secured to shaft section 347 (see FIG. 7A), which can be considered part of the distal tip. Shaft section 347 is coupled to bearing 351 (see FIG. 7D), through which the drive cable extends and rotates relative to. The distal tip also includes bearing 366 (see FIG. 7D), which can be a thrust bearing. Working portion 340 can be similar to or the same in some aspects to working portion 1104, even if not explicitly included in the description. In this embodiment, conduit 356 extends at least as far as ends of the impeller, unlike in working portion 1104. Either embodiment can be modified so that the conduit extends to a position as set forth in the other embodiment. In some embodiments, section 360 can be a tubular section instead of wound.

In alternative embodiments, at least a portion of any of the impellers herein may extend outside of the fluid lumen. For example, only a portion of an impeller may extend beyond an end of the fluid lumen in either the proximal or distal direction. In some embodiments, a portion of an impeller that extends outside of the fluid lumen is a proximal portion of the impeller, and includes a proximal end (e.g., see the proximal impeller in FIG. 6). In some embodiments, the portion of the impeller that extends outside of the fluid lumen is a distal portion of the impeller, and includes a distal end (e.g., see the distal impeller in FIG. 6). When the disclosure herein refers to impellers that extend outside of the fluid lumen (or beyond an end), it is meant to refer to relative axial positions of the components, which can be most easily seen in side views or top views, such as in FIG. 6.

A second impeller at another end of the fluid lumen may not, however, extend beyond the fluid lumen. For example, an illustrative alternative design can include a proximal impeller that extends proximally beyond a proximal end of the fluid lumen (like the proximal impeller in FIG. 6), and the fluid lumen does not extend distally beyond a distal end of a distal impeller (like in FIG. 7B). Alternatively, a distal end of a distal impeller can extend distally beyond a distal end of the fluid lumen, but a proximal end of a proximal impeller does not extend proximally beyond a proximal end of the fluid lumen. In any of the pump portions herein, none of the impellers may extend beyond ends of the fluid lumen.

Furthermore, any of these embodiments may be modified to include only a single expandable impeller disposed, e.g., at the distal end of the fluid lumen or at the proximal end of the fluid lumen.

While specific exemplary locations may be shown herein, the fluid pumps may be able to be used in a variety of locations within a body. Some exemplary locations for placement include placement in the vicinity of an aortic valve or pulmonary valve, such as spanning the valve and positioned on one or both sides of the valve, and in the case of an aortic valve, optionally including a portion positioned in the ascending aorta. In some other embodiments, for example, the pumps may be, in use, positioned further downstream, such as being disposed in a descending aorta.

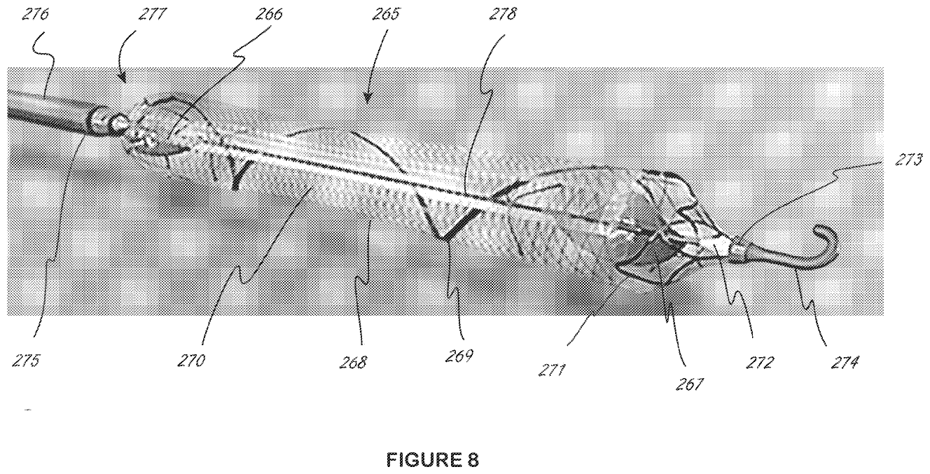

FIG. 8 illustrates a working portion that is similar to the working portion shown in FIG. 5. Working portion 265 includes proximal impeller 266, distal impeller 267, both of which are coupled to drive shaft 278, which extends into distal bearing housing 272. There is a similar proximal bearing housing at the proximal end of the working portion. Working portion also includes expandable member, referred to 270 generally, and conduit 268 that is secured to the expandable member and extends almost the entire length of expandable member. Expandable member 270 includes distal struts 271 that extend to and are secured to strut support 273, which is secured to distal tip 273. Expandable member 270 also includes proximal struts there are secured to a proximal strut support. All features similar to that shown in FIG. 5 are incorporated by reference for all purposes into this embodiment even if not explicitly stated. Expandable member 265 also includes helical tension member 269 that is disposed along the periphery of the expandable member, and has a helical configuration when the expandable member is in the expanded configuration as shown. The helical tension member 269 is disposed and adapted to induce rotation wrap upon collapse. Working portion 265 can be collapsed from the shown expanded configuration while simultaneously rotating one or both impellers at a relatively slow speed to facilitate curled collapse of the impellers due to interaction with the expandable member.

There are alternative ways to construct the pump portion to cause rotation of the expandable member upon collapse by elongation (and thus cause wrapping and collapse of the impeller blades). Any expandable member can be constructed with this feature, even in dual-impeller designs. For example, with an expandable member that includes a plurality of “cells,” as that term is commonly known (e.g., a laser cut elongate member), the expandable member may have a plurality of particular cells that together define a particular configuration such as a helical configuration, wherein the cells that define the configuration have different physical characteristics than other cells in the expandable member. In some embodiments the expandable member can have a braided construction, and the twist region may constitute the entire group of wires, or a significant portion (e.g., more than half), of the braided wires. Such a twisted braid construction may be accomplished, for example, during the braiding process, such as by twisting the mandrel that the wires are braided onto as the mandrel is pulled along, especially along the length of the largest-diameter portion of the braided structure. The construction could also be accomplished during a second operation of the construction process, such as mechanically twisting a braided structure prior to heat-setting the wound profile over a shaped mandrel.

Any of the conduits herein act to, are configured to, and are made of material(s) that create a fluid lumen therein between a first end (e.g., distal end) and a second end (e.g., proximal end). Fluid flows into the inflow region, through the fluid lumen, and then out of an outflow region. Flow into and out of the fluid lumen may be labeled herein as “F.” Any of the conduits herein can be impermeable. Any of the conduits herein can alternatively be semipermeable. Any of the conduits herein may also be porous, but will still define a fluid lumen therethrough. In some embodiments the conduit is a membrane, or other relatively thin layered member. Any of the conduits herein, unless indicated to the contrary, can be secured to an expandable member such that the conduit, where is it secured, can be radially inside and/or outside of the expandable member. For example, a conduit can extend radially within the expandable member so that inner surface of the conduit is radially within the expandable member where it is secured to the expandable member.

Any of the expandable member(s) herein can be constructed of a variety of materials and in a variety of ways. For example, the expandable member may have a braided construction, or it can be formed by laser machining. The material can be deformable, such as nitinol. The expandable member can be self-expanding or can be adapted to be at least partially actively expanded.

In some embodiments, the expandable member is adapted to self-expand when released from within a containing tubular member such as a delivery catheter, a guide catheter or an access sheath. In some alternative embodiments, the expandable member is adapted to expand by active expansion, such as action of a pull-rod that moves at least one of the distal end and the proximal end of the expandable member toward each other. In alternative embodiments, the deployed configuration can be influenced by the configuration of one or more expandable structures. In some embodiments, the one or more expandable members can deployed, at least in part, through the influence of blood flowing through the conduit. Any combination of the above mechanisms of expansion may be used.

The blood pumps and fluid movement devices, system and methods herein can be used and positioned in a variety of locations within a body. While specific examples may be provided herein, it is understood that that the working portions can be positioned in different regions of a body than those specifically described herein.

Blood pumps, such as any of the intravascular pumps herein, may benefit from having one or more fluid paths through which fluid can flow through the device. For example without limitation, blood pumps may benefit from having one or more fluid paths through which fluid can flow to perform any of these exemplary functions: cooling rotating components (e.g., a drive cable) to prevent their overheating; flushing small particulates that may break off rotating components (e.g., a drive cable) to prevent the rotating parts from being damaged by the small particulates; lubricating rotating components (e.g., one or more bearings), and preventing blood ingress into the pump (e.g., near or at a distal end of the pump). Fluid delivery through the one or more flow paths may provide any number of these functions.

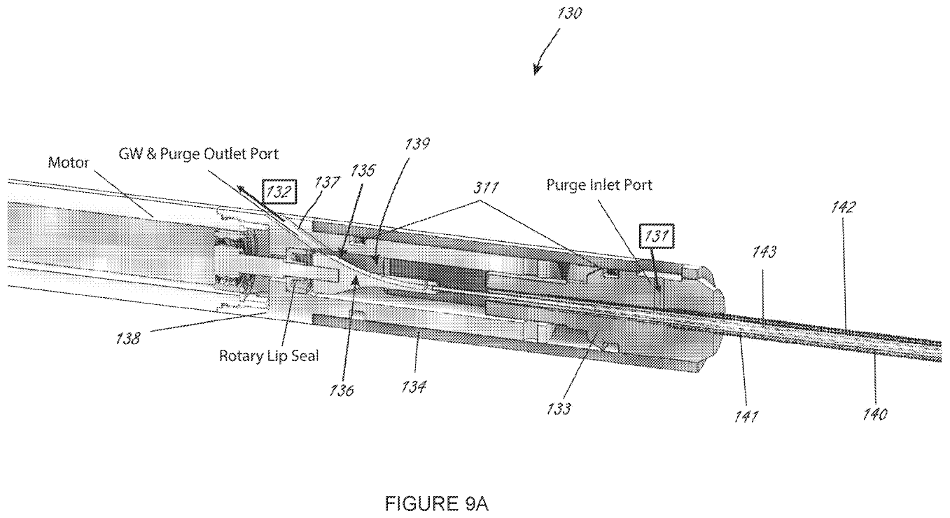

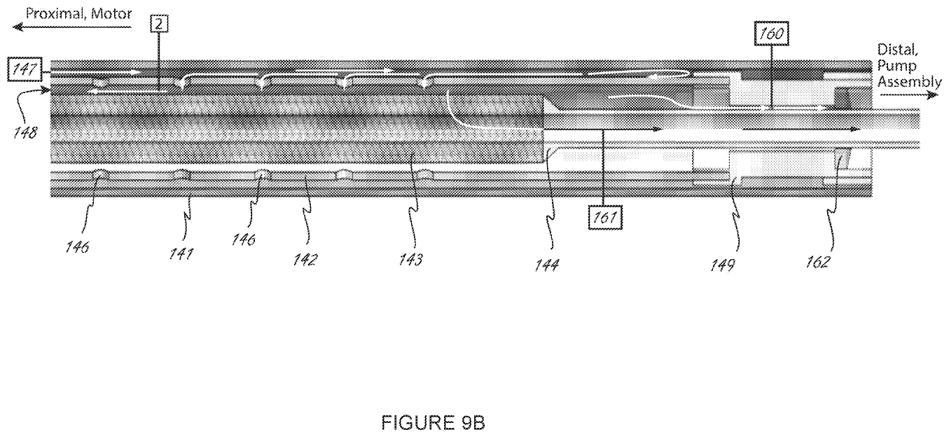

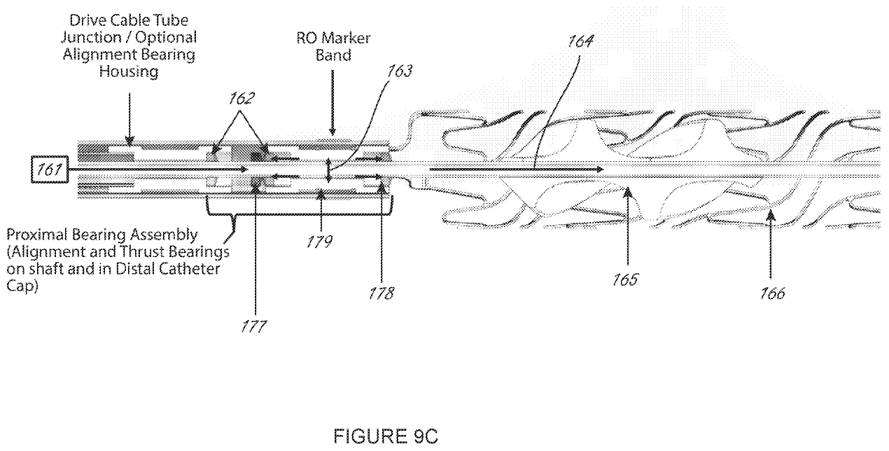

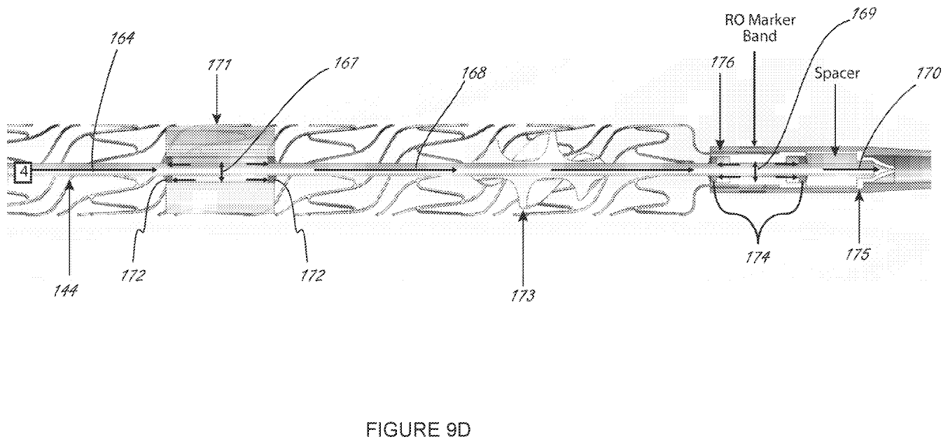

FIGS. 9A-D illustrate an exemplary embodiment of a fluid delivery system incorporated into an exemplary fluid pump (e.g., blood pump) with a fluid inlet port and a fluid outlet port. FIG. 9A illustrates a portion of the device that is proximal to the one or more impellers, and in this embodiment includes a proximal end of a catheter, a motor assembly that causes the rotation of a drive cable and impeller(s), a fluid inlet port, and fluid outlet port, and a guidewire port that allows access to a guidewire pathway or lumen.

FIG. 9B shows a region of the device that is distal to the region shown in FIG. 9A, but includes some of the catheter components that are shown in FIG. 9A. FIG. 9C shows a region of the device distal to the region in FIG. 9B, and FIG. 9D shows a region of the device distal to the view in FIG. 9C.

While FIGS. 9A-D illustrate different sections of an exemplary blood pumping device, it is understood that in alternative embodiments aspects of the system can vary. For example, in alternative embodiments the portion of the device with the impellers can vary and could only include a single impeller, or the expandable housing around the impeller could have a wide variety of configurations. It is understood that individual regions of the device can be incorporated by themselves into a variety of different types of blood pumps.

One aspect of this exemplary embodiment includes a guidewire access port that also functions as a fluid port, and in this embodiment a fluid outlet port. A motor sealing cap 138 includes, formed therein, a guidewire channel 137, including a guidewire port in a radially side surface that provides access from outside the device to channel 137. The motor sealing cap may be an optional component, and the guidewire channel 137 can alternatively be formed in a different part of the device (e.g., which may not function as a motor sealing cap). The device also includes drive cable coupler 135, which includes formed therein a guidewire channel 136, which is a portion of a guidewire pathway. Drive cable coupler 135 is rotated by the motor, and causes the rotation of drive cable 143, which causes rotation of the one or more impellers in the pump portion. These components are thus considered to be in rotational communication. Channel 137, including the guidewire port, is formed in the device and is not adapted to rotate when the motor rotates. Channel 136 formed in drive cable coupler 135 rotates when the drive cable coupler rotates. When drive cable coupler 135 is in the position shown in FIG. 9A, channel 137 is in alignment with channel 136, which allows a guidewire to be advanced through or removed from channel 137 and through channel 136. If the guidewire is being inserted, the guidewire can then be advanced further distally through the entire device and out a distal end, described in more detail below. As is also described in more detail below, the guidewire access port also acts as a fluid outlet port that allows return fluid to flow from return area 139 out of the outlet port.

One of the advantages of having the guidewire access port (part of channel 137) in the location that it is in this embodiment, is that, if needed after the pump portion has already been advanced to a location within the patient, a guidewire can be reinserted into the port and inserted all the way to and out of the distal end. Importantly, the guidewire can be reinserted without having to remove most of the device from the patient like with some rapid exchange designs, and without having to remove the motor assembly. This exemplary embodiment thus allows easy reentry of a guidewire without having to remove the motor assembly, and without having to remove the device from the subject.

Being able to reinsert the guidewire during use can be advantageous because it can, for example without limitation, allow for repositioning of the pump portion if desired or needed. For example, if the pump portion moves out of position relative to an anatomical landmark (e.g., an aortic valve), a guidewire may need to be inserted to safely reposition it relative to the anatomical landmark.

Because the guidewire path extends through a rotational component (e.g., drive cable coupler 135), it is important that the guidewire not be present in the guidewire path when the rotating component is active. The apparatuses herein can also include an automated sensing mechanism to detect the presence of the guidewire in the guidewire pathway, and/or a prevention mechanism that prevents the motor from being activated if the guidewire is in the lumen. For example without limitation, there could be a sensor that can selectively detect the presence of the guidewire in the guidewire pathway, and communicate that to a controller that prevents the motor from being activated.

In this embodiment there is a single fluid inlet channel or lumen 131 into which fluid can be delivered into the device. FIG. 9B illustrates a region of the device and illustrates different pathways the fluid can take after it has been delivered into the device. After the fluid is advanced into fluid inlet port channel 131 (which includes an inlet port), it travels through a space 147 between clean purge tube 141 and drive cable tube 142. This is considered clean input fluid. This pathway deadends at distal catheter cap 149. The fluid passes through the one or more apertures 146 formed in a distal region of drive cable tube 142 as shown in FIG. 9B, entering into an annular space between drive cable tube 142 and drive cable 143. Some of this fluid (optionally most of the fluid) returns in the proximal direction through this annular space, lubricating and cooling drive cable 143 and flushing potential particulate along its path. This return fluid continues to flow proximally and into area 139 shown in FIG. 9A, and continues to flow through channel 137 and out of the fluid port (which is also the guidewire access port). A fluid outlet port thus also functions as a guidewire access port in this embodiment.

While most of the fluid returns proximally to area 139, some of the fluid, after it passes through apertures 146, continues distally beyond the distal end of the drive cable 143. Some of the fluid follows proximal bearing path 160 through alignment bearing 162 to prevent blood ingress. Fluid flow along path 160 to bearing 162 can be controlled by, for example, controlling input flow pressure and throttling of the return fluid at the proximal region of the device.

Some of the fluid, after passing through apertures 146, will flow through drive cable 143, along path 161, and will continue distally through the device (e.g., through hypotube 144) and out holes to lubricate any rotating surfaces and to prevent blood ingress, described in more detail below. Guidewire lumen 145 is thus positioned to also function as a distal bearing fluid flow path.

Some fluid flows distally along path 161, as shown in FIG. 9C, and passes through holes along path 163, to lubricate one or more of bearings 162, thrust bearing 177, and alignment bearing 178. Some of the fluid continues distally in the direction of arrow 164 shown in FIG. 9C, through impeller 165 (which in this embodiment is a proximal impeller). Some of the fluid passes through apertures along path 167 to lubricate optional alignment bearings 172 that support central member 171, which may be any of the collapsible support members, including any of the central or intermediate members herein. Some fluid continues distally through the guidewire lumen in the direction of arrow 168, through optional distal impeller 173. Some fluid passes through holes along path 169 to lubricate bearings 174 that are distal to the distal impeller. Some of the fluid may also flow through valve 175 and out the distal end of the device, helping prevent blood ingress.

In this exemplary embodiment a single flow path flowing through a tubular member (path 161 that extends distally through guidewire lumen shown in FIG. 9B) leads to (is in fluid communication with) at least three distally located bearing lubricating fluid paths, 163, 167, and 169, which lubricated three axially spaced bearing regions. In some alternative embodiments, there may be a single bearing region that is lubricated, two bearing regions that are lubricated, or even more than three bearings regions that are lubricated, depending on the number of structures disposed within the expandable housing that require bearings and thus lubrication.

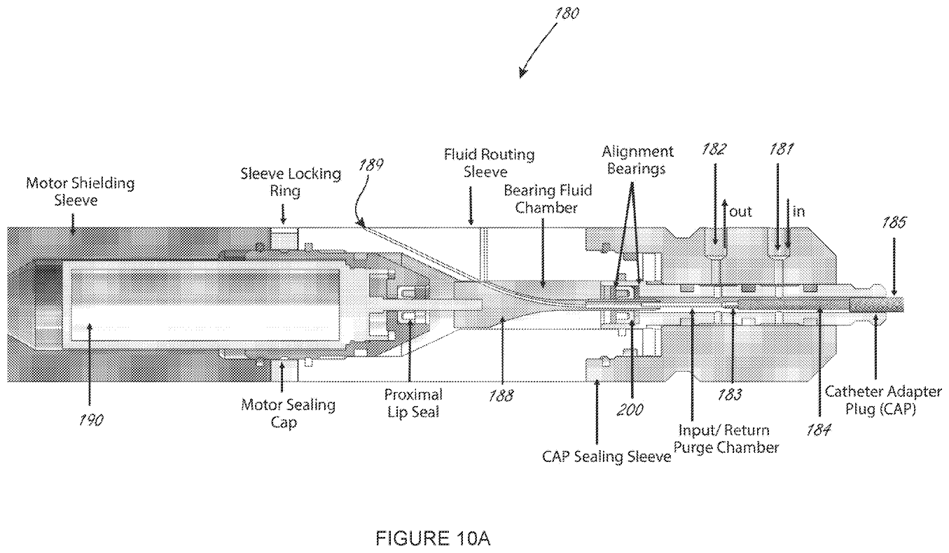

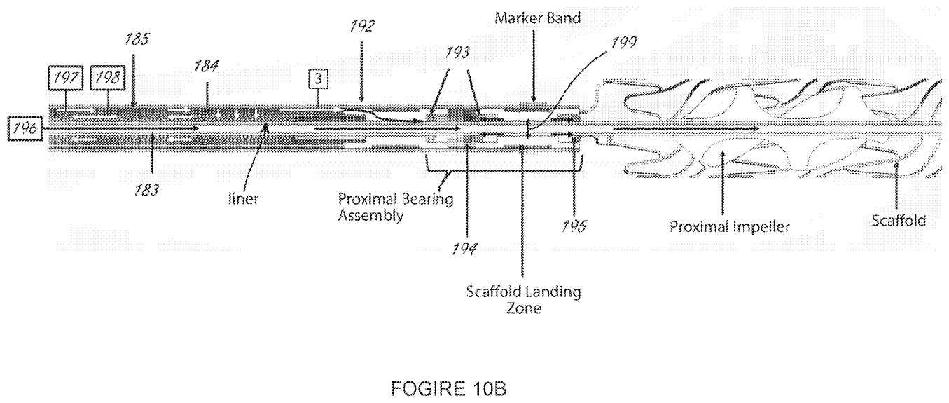

An exemplary method of using the device in FIGS. 9A-D includes inserting a guidewire near a target location (e.g., into a left ventricle via femoral artery access), then feeding the distal guidewire port over the guidewire and advancing the device over the guidewire towards the target location (e.g., an aortic valve). The method can also include removing the guidewire from the guidewire path, and coupling the proximal portion shown in FIG. 9A to a fluid inlet coupler and a fluid outlet coupler at the inlet and the outlet fluid locations, respectively. The motor can be activated to activate the one or more impellers. If the guidewire needed to be reinserted, the fluid out connector can be removed and a guidewire can be reinserted (e.g., for repositioning). The guidewire can then be removed, and the fluid outlet coupler can again be put into fluid communication with the guidewire pathway. These methods or any of them individually can be incorporated into the use of any of the suitable devices herein, such as the device in FIGS. 10A and 10B. Additionally, any of the steps in any of the other exemplary methods of use herein, such as those below, may be incorporated into a use of the blood pump in this embodiment.

FIGS. 10A and 10B illustrate an exemplary embodiment of a fluid delivery system incorporated into an exemplary fluid pump (e.g., blood pump) with a first flow path with a first fluid inlet port and a first fluid outlet port. In this embodiment, however, there is also a second fluid flow path that is not in fluid communication with the first flow path. The device 180 in FIGS. 10A and 10B is similar to that shown in the embodiment in FIGS. 9A-D, except in this embodiment the fluid path 161 from FIG. 9B does not originate as fluid that flows through the drive cable. In this embodiment the fluid flow path that includes the guidewire lumen (see fluid path 196 in FIG. 10B) is in fluid communication with a separate and second fluid inlet port 189, which is also located to function as a guidewire access port, as shown in FIG. 10A. Drive cable 183 has a drive cable liner 187 on its inner surface to seal off the distal bearing flow path 196 (through the guidewire lumen). In this embodiment the guidewire access port does not function as a fluid outlet, like in FIGS. 9A-D, but as a fluid inlet port, and thus still functions as a fluid port or fluid access.

The blood pump also includes a first fluid path that includes inlet port 181 and outlet port 182 as shown in FIG. 10A. This flow path is very similar to the path in FIGS. 9A-D, except that it does not include the path through the drive cable and hypotube (i.e., does not include the guidewire lumen). The fluid is advanced through port inlet port 181, flows distally along path 197 in FIG. 10B, which is between clean purge tube 185 and drive cable tube 184. This path deadends at a distal catheter cap, just as in the embodiment in FIGS. 9A-D. The fluid flows through holes in drive cable tube 184, and returns proximally in the annular space between drive cable tube 184 and drive cable 183. In this part of the path the fluid lubricates and cools the drive cable and flushes potential particulate along its path, carrying them proximally to fluid exit port 182 shown in FIG. 10A. Seal 200 prevents fluid from passing proximally to seal.

Fluid flowing through the first fluid path thus lubricates and cools the drive cable, as well as flushes potential particulates and returns to exit port 182. Fluid flowing through the second fluid path travels further distally through the system, and lubricates one or more distal bearings, just as in the embodiment in FIGS. 9A-D. For example, path 199 shown in Figure is the same as path 163 in FIG. 9C, which lubricates bearings in that bearing region. While not shown, the fluid flow path distal to the view shown in FIG. 10B can be exactly the same as in FIG. 9D, thus lubricating additional bearings, and optionally exiting through a valve at a distal end of the device. This second flow path can thus also prevent ingress of blood, which is described more fully in FIGS. 9A-D.

In any of the devices herein, the pump portion can include a distal end valve distal to the impeller to seal off the distal guidewire port after the guidewire is removed, but allows for guidewire reinserting therethrough.

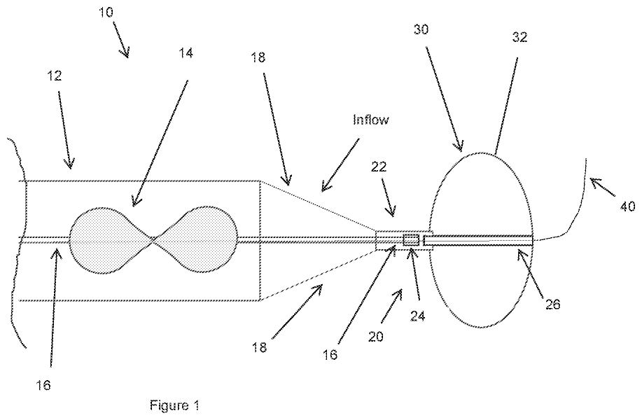

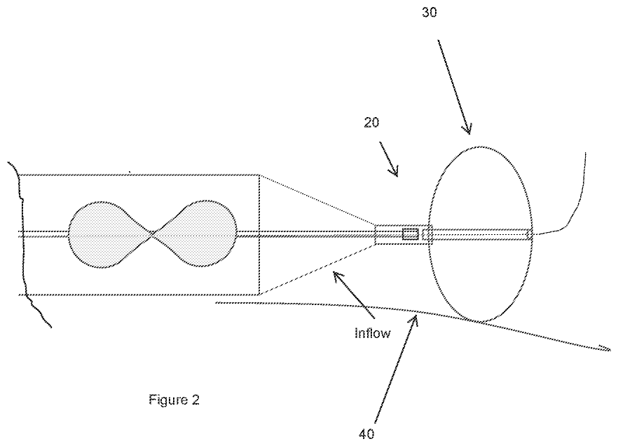

It may be desirable to include a distal inflatable member on an intravascular blood pump, such as the expandable intravascular blood pumps described above or in other intravascular blood pumps. FIG. 1 illustrates the expanded and deployed configuration of a distal portion of an exemplary catheter blood pump, such as those described above. The catheter-based intravascular blood pump includes pump portion 10 with a blood conduit 12, such as any of the collapsible and expandable shrouds or blood conduits described above. Pump portion 10 includes one or more expandable impellers 14 adapted to rotate to pump blood disposed within the blood conduit, such as in a proximal half of the blood conduit or a distal half of the blood conduit, or extending partially out of the blood conduit. In the example shown in FIG. 1, impeller 14 is generally closer to a pump inflow than a pump outflow. Pump portion 10 includes drive mechanism 16, which is coupled to at least one impeller and is in rotational communication therewith. Drive mechanism 16 may include any aspect of any of the drive mechanisms described above and may include one or more of a drive cable or a drive shaft. The drive mechanism 16 may be in communication with a motor, such as an external motor or on-board motor. Pump portion 10 also includes an inflow, which may comprise one or more struts 18 that define openings for blood to flow therethrough and into the blood conduit, such as any of the struts described above.

Pump portion 10 may include a distal pump region 20, which is generally distal to blood conduit 12 and proximate to the pump inflow. In this example the distal pump region 20 is distal to the inflow. In this example, inflatable member 30 is coupled to distal pump region 20. FIG. 1 shows inflatable member in an inflated state after being inflated with inflation fluid, such as purge fluid or fluid from an external fluid source. As shown, the inflated diameter of inflatable member 30 is greater than the expanded diameter of the expanded blood conduit 12.

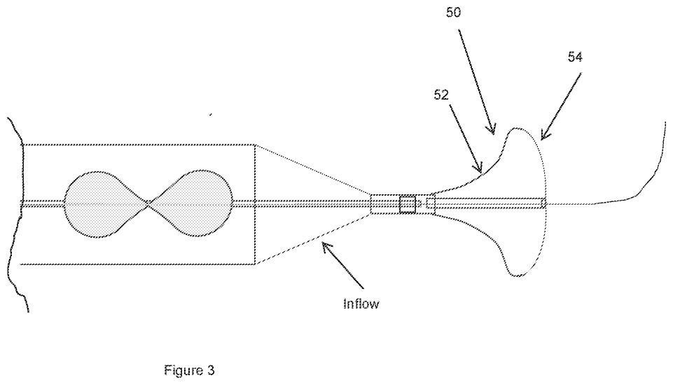

FIG. 2 illustrates the pump portion from FIG. 1, but only inflatable member 30 and distal pump region 20 from the blood pump are labeled. All of the other components in FIG. 2 may be the same as the components in FIG. 1. FIG. 2 show inflatable member 30 in an inflated state after being delivered in an un-inflated state, such as within a delivery sheath. As shown, inflatable member has a configuration and volume in this state that maintains a spacing between distal pump region 20, particularly the inlet openings of distal pump region 20, and tissue 40 that is adjacent to distal pump region 20. In this example, tissue 40 may be a left ventricular wall. Inflating the inflatable member 30 as shown may also prevent the left ventricular wall or other tissue from being pulled into the inflow during pump operation.

In the merely exemplary embodiment of FIGS. 1 and 2, distal pump region 20 may include a central hub portion 22 (see FIG. 1), to which exemplary distal struts 18 may be coupled or connected. A distal end of drive mechanism 16 may extend into central hub portion 22 as shown, and which may be disposed within bearing 24 to facilitate rotation of drive mechanism 16 relative to central hub portion 22. In the example shown in FIG. 1, a proximal end region of the inflatable member 30 is coupled to a distal region of central hub portion 22. An elongate member 26 with a lumen therein is coupled within a recessed region of hub portion 22 and extends distally therefrom, as shown. In this example, the drive mechanism and elongate member 26 form part of a guidewire pathway, through which a guidewire 40 may be disposed.

An elongate member 26 may be more flexible than central hub portion 22, which may allow the inflatable member 30 and elongate member 26 to deflect laterally (up and down in the figure) without causing central hub 22 to deflect. This may help maintain a tip gap between blades of impeller 14 and an inner wall of blood conduit 12, as well as help prevent the blades of impeller 14 from hitting the blood conduit 12.

FIG. 1 illustrates an exemplary inflatable member that is coupled to an axially extending element that forms part of a guidewire lumen. In alternatives to the blood pump shown in FIG. 1, the pump may exclude an elongate element 26, and instead central hub portion 22 may extend further distally. In these alternatives, the distal end of inflatable member 30 may be coupled to the central hub portion 22 rather than to an elongate element extending from a central hub.

In any of the embodiments herein, the inflatable member may comprise an elastic material, such as a polymeric material.

In any of the embodiments herein, the inflatable member may comprise an inelastic or primarily inelastic material, which is optionally preformed to have a desired inflated configuration so that inflation of the internal volume inflates the inflatable member toward the pre-formed shape. For example, a pre-formed inflated configuration may be spherical. In some embodiments, a pre-formed inflated configuration may be ovoid. In some embodiments, a pre-formed inflated configuration may be toroidal.

In any of the embodiments herein, the inflatable member may have a proximal region with a surface configured to transition blood flow towards the pump inflow. FIG. 3 illustrates an exemplary blood pump with inflatable member 50 in an inflated state with a proximal region 52 with a surface configured to transition blood flow towards the pump inflow. Any feature of the blood pump in FIG. 1 may be incorporated into the pump of FIG. 3, even though they are not labeled. FIG. 3 illustrates an inflatable member with a proximal region 52 that comprises a curved surface having a radially outer dimension decreasing in a proximal direction. FIG. 3 illustrates an inflatable member with a proximal region 52 that comprises a concave surface. The proximal region of any inflatable member herein may have a surface configured to entrain flow towards the pump portion inflow. Once again, the largest diameter of the inflatable member 50 is larger than the expanded diameter of the pump's blood conduit, as shown in FIG. 3.

While not shown, any of the inflatable members herein may comprise a plurality of apertures therein. The apertures may be small enough to allow the inflatable member to be pressurized and inflated, yet allow inflation fluid to exit the internal volume through the apertures. In some embodiments the inflatable member comprises a weeping balloon. The inflatable members herein may be in fluid communication with a purge fluid source such that they are inflated with fluid that acts to purge one or more components in the system. For example, the inflatable members herein may be in fluid communication with a purge fluid pathway in the catheter, such as any of the purge fluid pathways described above.

In some embodiments, an inflatable member inflation fluid pathway includes a lumen within a rotatable drive mechanism, such as drive mechanism 16, which may also be a guidewire lumen as well as a purge fluid pathway. Inflation fluid may pass from within a lumen within drive mechanism 16 and through a pathway within the distal pump portion 20 and into the internal volume within inflatable member 30 to inflate the inflatable member. The fluid may also lubricate bearing 24 within the central hub 22. Part of the inflation fluid pathway may include a volume between central hub 20 an elongate member 26.

In any of the embodiments herein, the blood conduit and one or more impellers may be non-collapsible. In any of the embodiments herein, the blood conduit and one or more impellers may be adapted to be collapsed and expanded, such as any of the shrouds and impellers in the Appendix section.

In any of the embodiments herein, the inflatable member may have a separate inflation fluid pathway that is not in fluid communication with a purge fluid (clean or waste) pathway. In these embodiments, the inflatable member may be in fluid communicated with a separate and dedicated inflation fluid reservoir.

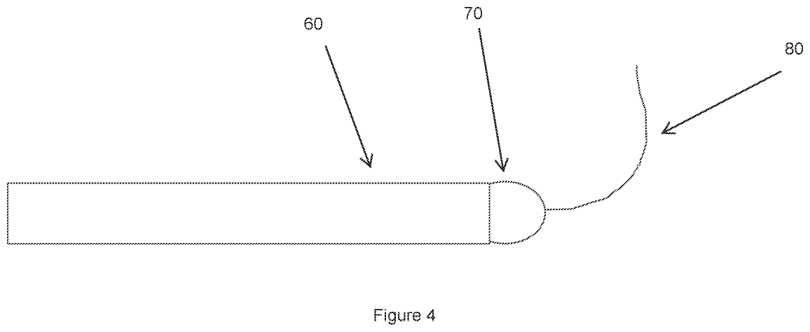

The inflatable members herein may optionally be adapted and configured for use as a cover or seal for a distal end of a delivery sheath for delivery. When medical devices and implants are delivered intravascularly, the distal end of an outer sheath is generally covered or sealed off to prevent blood from entering into the space within the sheath. In any of the embodiments herein, the inflatable member may function to cover the distal end of the sheath to prevent blood from entering therein. FIG. 4 illustrates an example of sheath 60 and inflatable member 70 covering the distal end sheath 60. While not shown, a catheter blood pump is disposed within the sheath 60. A catheter blood pump within sheath 60 may be collapsible or non-collapsible. Guidewire 80 is disposed within the blood pump and extending distally relative to inflatable member 70.

Inflatable members herein may be inflated to a partially or non-fully inflated state to adapt them to cover the distal end of outer sheath, as is shown in FIG. 4. For tracking or delivery, the inflatable member may be partially inflated, but not to a fully inflated state, an example of which is shown in FIG. 4. In this state the inflatable member can prevent blood from entering into the sheath, but is not inflated enough to interfere with tracking and delivery. In some embodiments the catheter blood pump inflatable members herein may have at least two inflation states, a first state for delivery/tracking through the vasculature, and a second state for spacing the distal end or inflow from adjacent. The second state may be more inflated than the first state and may have a larger radially outermost dimension compared to the first state. For example, FIG. 4 may show a partially inflated inflatable member 70, and FIGS. 1-3 may show a second state of the inflatable members. In some embodiments the system may be adapted to have discrete inflation levels for first and second inflation states. For example, a controller (e.g., algorithm or computer executable method) may be adapted to automatically control inflation levels for a partially or non-fully inflated state (example in FIG. 4) for tracking and delivery to seal off the sheath distal end, and a fully inflated state for spacing (examples in FIGS. 1-3).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An intravascular blood pump, comprising:
an expandable blood conduit having an unexpanded delivery diameter and an expanded deployed diameter;
one or more expandable impellers disposed at least partially in the blood conduit; and
an inflatable member disposed distal to the blood conduit, the inflatable member having an uninflated delivery configuration and an inflated configuration, the inflatable member having a diameter in the inflated configuration that is greater than the expanded deployed diameter of the blood conduit.

2. The intravascular blood pump of claim 1, wherein the one or more impellers comprises an impeller disposed at a distal end of the blood conduit.

3. The intravascular blood pump of claim 1, further comprising a guidewire lumen extending through the inflatable member.

4. The intravascular blood pump of claim 3, further comprising an elongate member extending distally from a hub at a distal end of the blood conduit, the guidewire lumen extending through the hub and the elongate member.

5. The intravascular blood pump of claim 4, wherein the elongate member is more flexible than the hub.

6. The intravascular blood pump of claim 1, wherein the inflatable member comprises an inelastic material.

7. The intravascular blood pump of claim 1, wherein the inflatable member has a preformed shape.

8. The intravascular blood pump of claim 7, wherein the preformed shape is spherical.

9. The intravascular blood pump of claim 7, wherein the preformed shape is ovoid.

10. The intravascular blood pump of claim 7, wherein the preformed shape is toroidal.

11. The intravascular blood pump of claim 1, wherein the inflatable member is adapted and configured to be partially inflated during intravascular delivery to seal a distal end of a delivery sheath.
